(19) United States
(12) Patent Application Publication
(10) Pub. No.: US 2015/0272844 A1
(43) Pub. Date: Oct. 1, 2015

(54) Topical skin care formulations

(71) Applicant: Louise Holyfield, Dallas, TX (US)

(72) Inventor: Louise Holyfield, Dallas, TX (US)

(21) Appl. No.: 14/545,563

(22) Filed: May 21, 2015

Related U.S. Application Data
(63) Continuation-in-part of application No. 13/999,849, filed on Mar. 27, 2014, now Pat. No. 9,060,946.

(51) Int. Cl. A61K 8/37; A61K 8/34; A61K 8/35; A61Q 19/04

(52) U.S. Cl. CPC A61K 8/37; A61Q 19/04; A61K 8/347; A61K 8/35

(57) ABSTRACT

A white, color stable, sunless tanning formulation containing vanillin and dihydroxyacetone for tanning, and octyl salicylate plus octyl methoxycinnamate for color stabilization.

### FIELD OF INVENTION

This invention relates to chemical compositions, and more particularly to topical self-tanning, sunless tanning, anti-aging, anti-oxidant skin care formulations and methods of use.

### BACKGROUND OF THE INVENTION

More than 3.5 million new cases of skin cancers are diagnosed each year in the United States. Young adults under the age of 25 face the biggest risk. Skin cancer is the most common form of cancer in the United States.

Although there are many alarms being sounded regarding the danger of overexposure to the sun and the use of tanning beds, it appears it is falling on deaf ears as many individuals tan to the point of addiction, believing they are more attractive with tanned skin.

Since world population grows exponentially, there is every indication that people, especially young individuals, will continue to seek ways in which to tan their skin, although many know the inherent danger therein.

The DHA self-tanning, sunless tanning products in the marketplace are dark in color as the result of dyes and stains used in the formulation so that the user can get a so-called “instant tan”. The product is messy and irreversibly stains everything it comes in contact with.

Product whiteness represents cleanliness and purity to the consumer and is a commercially preferred color for products that are used on the face and body.

Vanillin is a natural ingredient that is effective for tanning the skin, and it also contains anti-aging and anti-oxidant benefits. Although initially white, vanillin degrades to various shades of brown within a few days, when used in an aqueous emulsion.

### SUMMARY OF THE INVENTION

A white, color-stable formulation containing vanillin and Dihydroxyacetone (DHA) in a self-tanning, sunless tanning, topical formulation that tans the skin quickly without the use of dyes or stains.

This product formulation does not stain clothing, linens, towels, etc. The formulation works at a cellular level and therefore does not require that the skin be exfoliated prior to the application of the product.

Prior self-tanning, sunless tanning methods usually include the step of exfoliating the skin before applying the product. A method that avoids this step is provided.

My previous research has achieved stable whiteness by adding octyl salicylate and octyl methoxycinnamate to sunless tanning products containing vanillin, with or without other tanning agents. See U.S. application Ser. No. 13/999,849 incorporated herein by reference.

More recent research has led me to the discovery that stable whiteness, and a more attractive, more natural-looking tan can be achieved with formulations of the prior application, only by using the correct ratios of vanillin to DHA, in aqueous emulsions.

DHA as the sole tanning agent in such a formulation cannot give the user a natural-looking tan. Instead, the resulting tan always has a distinctive yellow tint. Vanillin as the sole tanning agent in such a formulation cannot give the user a natural-looking tan. Instead, the resulting tan always has a distinctive brown tint.

The invention provides a white, color-stable topical self-tanning, sunless tanning anti-aging, anti-oxidant aqueous emulsion that enables a quick, superior tan, does not contain dyes or colorants, and does not stain clothing, linens, or other fabrics.

Another aspect of the invention provides a white, color-stable topical self-tanning, sunless tanning, anti-aging, anti-oxidant aqueous emulsion to be used not only by individuals, but also by massage therapists for giving clients a sunless tan and a massage at the same time. This has never been acceptable before, because sunless tanning lotions have always caused severe staining of clothing, sheets and other fabrics.

Another aspect of the invention provides a white, color-stable topical self-tanning, sunless tanning, anti-aging, anti-oxidant aqueous emulsion that is superior in its ability to tan the skin uniformly, without streaking or fading unevenly.

Another aspect of the invention provides a white, color-stable topical self-tanning, sunless tanning, anti-aging, anti-oxidant aqueous emulsion does not require exfoliation of the skin prior to application.

### EXAMPLES

A. 10% by wt. Vanillin
   3% by wt. Dihydroxyacetone (DHA)
   1.5% by wt. Octyl Salicylate
   1.5% by wt. Octyl Methoxycinnamate
   84% by wt. white emulsion, including water and oil, and emulsifier
B. 15 by wt. Vanillin
   3% by wt. Dihydroxyacetone (DHA)
   1.5% by wt. Octyl Salicylate
   1.5% by wt. Octyl Methoxycinnamate
   79% by wt. white emulsion, including water and oil, and emulsifier
C. 15% by wt. Vanillin
   4% by wt. Dihydroxyacetone (DHA)
   1.5% by wt. Octyl Salicylate
   1.5% by wt. Octyl Methoxycinnamate
   78% by wt. white emulsion, including water and oil, and emulsifier
D. 18% by wt. Vanillin
   5% by wt. Dihydroxyacetone (DHA)
   1.5% by wt. Octyl Salicylate
   1.5% by wt. Methoxycinnamate
   74% by wt. white emulsion, including water and oil, and emulsifier

The percentage range of Vanillin and DHA varies in a formulation, depending on an individual's tan color preference of light, medium, or dark. Vanillin is used in the range of 1.0% to 22 wt %, and preferably 5% to 18 wt %. DHA is used in the range of 1.0% to 5 wt %, and preferably 3% to 5 wt %. The ratio of vanillin to DHA is from 2:1 to 6:1 and preferably 3:1 to 5:1.

Octyl salicylate is used in the range of 1.0% to 3 wt %, and preferably 1.5 to 3 wt %. Octyl methoxycinnamate is used in the range of 1.0% to 3 wt % and preferably 1.5% to 3 wt %

An “aqueous emulsion” is defined as any suitable oil dispersed in water. For details on the preparation of suitable emulsions, see U.S. Pat. No. 6,906,106 incorporated herein by reference. White cosmetic emulsions are readily available, but they have never remained white when mixed with vanillin. The present invention solves that problem.

For a suitable list of vanillin derivatives useful in the formulations of the present invention, see U.S. Pat. No. 7,935,331 incorporated herein by reference.

The combination of DHA and Vanillin enables a tan to begin to develop on the skin in a shorter period of time than when each is used alone. For example, a 3 to 1 ratio of Vanillin to DHA used in combination enables the development of color on the skin to begin in about 30 minutes after application, and continue to fully develop to the desired color in 4 to 6 hours. By comparison, it takes DHA alone one or two hours to begin tanning; and vanillin alone takes even longer.

Vanillin alone gives the skin a somewhat brownish looking tan whereas DHA alone tends to give the skin a slightly yellowish tone. However, when used in combination the Vanillin overrides the yellow tone and the results are a longer lasting more natural looking ₜan color on the skin.

1. A white, color-stable sunless tanning formulation comprising:
an effective amount of vanillin for tanning the skin,
an effective amount of dihydroxyacetone for tanning the skin,
said amounts of vanillin and dihydroxyacetone having the necessary ratio to give the user a natural-looking tan,
a white emulsion comprising water, oil, and an emulsifier, and
effective amounts of octyl salicylate and octyl methoxycinnamate for color stabilization.

2. A method comprising the step of applying the formulation of claim 1 to the skin, for the purpose of obtaining a sunless tan.

3. A method for preserving whiteness in a white formulation containing vanillin and dihydroxyacetone in a white emulsion, comprising the step of adding octyl salicylate and octyl methoxy-cinnamate to said formulation, for color stabilization.

4. A method of skin care as in claim 2 completed during massage therapy, to obtain the benefits of sunless tanning and massage therapy, all at the same time.

5. The formulation of claim 1 wherein the amount of vanillin is 1% to 22 wt %.

6. The formulation of claim 1 wherein the amount of DHA is 1% to 5 wt %.

7. The formulation of claim 1 wherein the ratio of vanillin to DHA is from 2:1 to 6:1.
